(19) United States
(12) Patent Application Publication
(10) Pub. No.: US 2025/0346430 A1
(43) Pub. Date: Nov. 13, 2025

(54) SYSTEMS AND METHODS FOR MONITORING STORAGE SITES

(71) Applicant: Schlumberger Technology Corporation, Sugar Land, TX (US)

(72) Inventors: Oguzhan Guven, Rosharon, TX (US); Yuxiang Liu, Rosharon, TX (US); Arunkumar Arumugam, Rosharon, TX (US); Karthik Kundapur, Rosharon, TX (US)

(21) Appl. No.: 19/202,192

(22) Filed: May 8, 2025

Related U.S. Application Data
(60) Provisional application No. 63/644,219, filed on May 8, 2024.

(51) Int. Cl. B65G 5/00; E21B 33/138; E21B 47/117; G01M 3/04; G01N 33/00

(52) U.S. Cl. CPC B65G 5/005; E21B 47/117; G01M 3/04; G01N 33/004; E21B 33/138

(57) ABSTRACT

A monitoring system for monitoring a geological formation includes a casing of an injection well, wherein the casing is configured to extend through an open hole of the geological formation to define an annular space between the casing and the open hole, and wherein the casing includes a mandrel circumscribing at least a portion of the casing, and one or more monitoring lines configured to monitor the geological formation for the presence of carbon dioxide, wherein the one or more monitoring lines are at least partially integrated with the mandrel.

### CROSS-REFERENCE TO RELATED APPLICATION

The present application is a U.S. Non-Provisional Patent Application claiming benefit of U.S. Provisional Patent Application No. 63/644,219, entitled “CCS ZONAL INTEGRITY AND SEALING SYSTEMS”, filed May 8, 2024, which is herein incorporated by reference.

### FIELD

The present disclosure relates to storing and monitoring the storage of undesirable gases (e.g., carbon dioxide).

### BACKGROUND

Industrial plants often combust hydrocarbon-containing materials, such as coal, oil, and natural gas, to generate heat and/or power for various equipment and/or processes. Flue gas is generated as a byproduct of the combustion process and may be treated prior to being released into the atmosphere. For example, carbon capture, utilization, and storage (CCUS) refers to a set of technologies and processes designed to capture carbon dioxide (CO₂) emissions from industrial processes or power generation, utilize the captured CO₂ in various applications, and store the CO₂ underground to limit it from entering the atmosphere and contributing to climate change. In conventional CCUS operations, monitoring and verification of CO₂ storage often involves the installation and maintenance of dedicated monitoring wells that are used to assess the integrity of the storage site and to detect any potential leaks of the stored CO₂. However, the installation and maintenance of these dedicated monitoring wells can add significant costs to CCUS projects.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In an embodiment, a monitoring system for monitoring a geological formation includes a casing of an injection well, wherein the casing is configured to extend through an open hole of the geological formation to define an annular space between the casing and the open hole, and wherein the casing includes a mandrel circumscribing at least a portion of the casing, and one or more monitoring lines configured to monitor the geological formation for the presence of carbon dioxide, wherein the one or more monitoring lines are at least partially integrated with the mandrel.

In another embodiment, a monitoring system for a well includes one or more monitoring lines configured to monitor the well, a mandrel configured to circumscribe a casing of the well, wherein the mandrel defines a recess, and an insert configured to cooperate with the recess of the mandrel to retain the one or more monitoring lines within the mandrel.

In another embodiment, a monitoring system for a well includes one or more monitoring lines configured to monitor the well, a mandrel configured to circumscribe a casing of the well, wherein the mandrel defines a recess, one or more centralizers positioned within the recess and configured to centralize the one or more monitoring lines within the recess, and an injectable binding agent configured to be injected into the recess to retain the one or more monitoring lines within the recess of the mandrel.

### BRIEF DESCRIPTION OF THE DRAWINGS

The subject disclosure is further described in the detailed description which follows, in reference to the noted plurality of drawings by way of non-limiting examples of the subject disclosure, in which like reference numerals represent similar parts throughout the several views of the drawings, and wherein:

FIG. 1 is a schematic diagram of an embodiment of carbon capture storage system having an injection well that intersects a geological formation, in accordance with aspects of the present disclosure;

FIG. 2 is a cross-sectional view of an embodiment of a monitoring system having monitoring lines unsecured to a casing, in accordance with aspects of the present disclosure;

FIG. 3 is a partial cross-sectional view of an embodiment of a monitoring system having monitoring lines secured to a casing via an adhesive, in accordance with aspects of the present disclosure;

FIG. 4 is a partial cross-sectional view of an embodiment of a monitoring system having monitoring lines secured to a casing via an adhesive, in accordance with aspects of the present disclosure;

FIG. 5 is a schematic view of an embodiment of a monitoring system having monitoring lines integrated with and/or incorporated into a casing, in accordance with aspects of the present disclosure;

FIG. 6 is a cross-sectional view of an embodiment of the monitoring system of FIG. 5 having monitoring lines integrated with and/or incorporated into a casing, in accordance with aspects of the present disclosure;

FIG. 7 is a schematic view of an embodiment of a monitoring system having monitoring lines integrated with and/or incorporated into a casing, illustrating a close-up perspective view of a portion, in accordance with aspects of the present disclosure;

FIG. 8 is an exploded perspective view of an embodiment of the monitoring system of FIG. 7, in accordance with aspects of the present disclosure;

FIG. 9 is a cross-sectional view of an embodiment of the monitoring system of FIG. 7 having monitoring lines integrated with and/or incorporated into a casing, in accordance with aspects of the present disclosure;

FIG. 10 is a schematic view of an embodiment of a monitoring system having monitoring lines integrated with and/or incorporated into a casing, illustrating a close-up perspective view of a portion, in accordance with aspects of the present disclosure;

FIG. 11 is a perspective view of an embodiment of the monitoring system of FIG. 10, in accordance with aspects of the present disclosure;

FIG. 12 is a perspective view of an embodiment of the monitoring system of FIG. 10, in accordance with aspects of the present disclosure;

FIG. 13 is a cross-sectional view of an embodiment of the monitoring system of FIG. 10, in accordance with aspects of the present disclosure; and

FIG. 14 is a schematic view of an embodiment of a clamp for a monitoring system, in accordance with aspects of the present disclosure.

### DETAILED DESCRIPTION

The particulars shown herein are by way of example and for purposes of illustrative discussion of the embodiments of the subject disclosure only and are presented in the cause of providing what is believed to be the most useful and readily understood description of the principles and conceptual aspects of the subject disclosure. In this regard, no attempt is made to show structural details in more detail than is necessary for the fundamental understanding of the subject disclosure, the description taken with the drawings making apparent to those skilled in the art how the several forms of the subject disclosure may be embodied in practice. Furthermore, like reference numbers and designations in the various drawings indicate like elements.

Carbon capture, utilization, and storage (CCUS) refers to a set of technologies and processes designed to capture carbon dioxide (CO₂) emissions from industrial processes or power generation, utilize the captured CO₂ in various applications, and store the CO₂ to limit the CO₂ from entering the atmosphere and contributing to climate change. For example, CO₂ may be captured from various sources and/or processes and transported to a location for injection into an underground geological formation (e.g., storage site). The underground geological formation may include various layers with differing characteristics that enable the geological formation to store the CO₂ in the subsurface rock. For example, the geological formation may include one or more porous layers (e.g., permeable layer, porous reservoir, deep saline formation or layer, deep saline aquifer, depleted hydrocarbon formation or layer), one or more sealing rock layers (e.g., caprocks, impermeable layer), as well as additional layers (e.g., drinking aquifer). The CO₂ may be injected into the one or more porous layers (e.g., into a storage site), and the one or more sealing layers may be positioned above and/or below the one or more porous layers to seal the one or more porous layers, thereby preventing carbon dioxide injected into the porous layers from reaching the additional layers and/or the atmosphere.

The storage operations may further include monitoring the well and/or the storage site for extended periods of time to ensure that the integrity of the storage site is maintained and/or to identify potential leaks of the stored CO₂ that may affect the various layers positioned above and/or below the storage site. For example, in traditional CCUS operations, monitoring and verification of CO₂ storage often involves the installation and maintenance of dedicated monitoring wells, which may be distinguishable from injection wells in that the dedicated monitoring wells are not configured to inject CO₂ into the geological reservoir. Rather, these dedicated monitoring wells are drilled through the geological formation within a threshold distance from the injection well and are used to assess the integrity of the storage site and/or to detect any potential leaks of the stored CO₂. For example, the dedicated monitoring wells may monitor the one or more sealing layers for the presence of CO₂, which may be indicative of a potential issue at the storage site. Unfortunately, the installation and maintenance of these dedicated monitoring wells can add significant costs to CCUS projects and/or operations.

Additionally, or alternatively, traditional CCUS operations may employ monitoring lines along a length of a casing of an injection well to monitor and/or verify the injection well (e.g., assess the integrity of the storage site). The monitoring lines may be positioned in the annular space extending between the casing and the geological formation from the surface to the open hole. To ensure the integrity of the storage site and/or to limit potential leaks of CO₂ from the storage site, cementing operations may be performed, whereby cement is injected into the annular space to seal the annular space along at least a portion of the length of the wellbore. Unfortunately, the monitoring lines of traditional systems may be installed and/or positioned within the annular space in a manner that interferes with cementing operations (e.g., cement flow), thereby reducing or preventing bonding of the cement with the open hole and the casing. As a result, traditional systems may be susceptible to CO₂ leakage along the injection well (e.g., along the annular space between the casing and the open hole), which may be undesirable.

Accordingly, present embodiments are directed toward monitoring systems that incorporate monitoring lines (e.g., fiber optic, electric, and/or optical telemetry cables, monitoring cables, other monitoring lines) along a length of an injection wellbore to monitor and verify the integrity of a storage site fluidly coupled to the injection wellbore. More particularly, present embodiments are directed toward monitoring systems that are installed and/or configured in a manner that provides substantially improved or optimal cementing conditions, thereby limiting and/or blocking a tendency of CO₂ to leak through an annular space between a casing of the injection wellbore and the open hole. For example, the monitoring systems discussed herein may include various components and/or features that enable the monitoring lines to be at least partially integrated with and/or incorporated into the borehole casing of an injection well, thereby providing substantially improved or optimal conditions for cementing operations. That is, the monitoring systems discussed herein may include one or more sections that at least partially incorporate and/or integrate the monitoring line(s) with the borehole casing, thereby providing increased space between the outer diameter of the borehole casing and the open hole (e.g., geological formation) and/or providing surfaces (e.g., smooth surfaces, surfaces flush with the borehole casing) that facilitate cementing operations. In some embodiments, the one or more sections that at least partially incorporate and/or integrate the monitoring line(s) with the borehole casing provide unobstructed lengths that facilitate cementing operations. That is, the cement may fill the space between the outer diameter of the borehole casing and the open hole for a mandrel length without interference of any monitoring lines to the cementing operation. The increased space and/or the surfaces (e.g., smooth surfaces) provided by integrating the monitoring line with the borehole casing may improve cementing conditions, thereby improving the efficiency and/or efficacy of a cementing operation. As a result, the integrity of the storage site is improved and the likelihood of potential CO₂ leakage is reduced. In certain embodiments, the borehole casing may be designed and/or configured such that the one or more sections that include the monitoring lines integrated with the borehole casing align with the one or more sealing layers of the geological formation. For example, it may be particularly beneficial to provide substantially improved or optimal cementing conditions along the injection well at positions corresponding to the one or more sealing layers to ensure the integrity of the storage site.

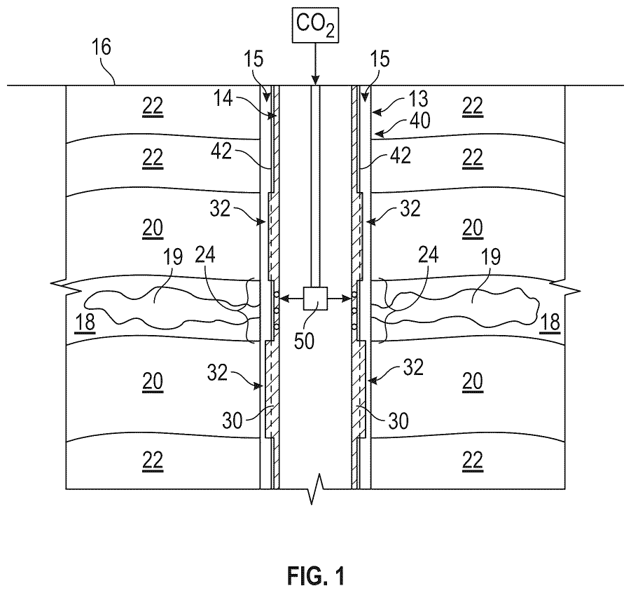

With the preceding in mind, FIG. 1 is a schematic view of an embodiment of a carbon capture storage system (CCSS) 10 for carbon capture, utilization, and storage (CCUS) operations. The CCSS 10 may include various components configured to enable the storage of carbon dioxide (CO₂) in a geological formation 12 of the CCSS 10, which may correspond to a volume of subsurface rock (e.g., subterranean formation) that contains various layers (e.g., rock layers, porous layers, aquifers, impermeable layers). For example, the CCSS 10 may include a well 14 (e.g., geological well, injection well) drilled from the surface 16 into and through the geological formation 12 to form an open hole 13 within the geological formation 12, where the well 14 intersects the various layers in the subsurface rock of the geological formation 12. In certain embodiments, the well 14 may correspond to an injection well configured to inject and/or direct undesirable fluid (e.g., carbon dioxide) into one or more of the layers of geological formation 12, thereby enabling the geological formation 12 to effectively store (e.g., permanently store) the undesirable fluid within the subsurface rock of the geological formation 12.

Each of the various layers may include different characteristics that enable the geological formation 12 to effectively store (e.g., permanently store) CO₂ introduced into the formation 12 (e.g., via the injection well 14). For example, the geological formation 12 may include an injection layer 18 (e.g., storage layer, porous layer, receiving layer), one or more sealing layers 20 (e.g., impermeable layers, caprock layers), and one or more additional layers 22. The injection layer 18 may correspond to a portion of the geological formation 12 that is capable of receiving CO₂. For example, a permeability and/or porosity of the injection layer 18 may enable CO₂ to be injected and contained within the injection layer 18. In certain embodiments, the injection layer 18 may correspond to a deep saline aquifer, a depleted hydrocarbon reservoir, a basalt formation, and the like. In certain embodiments, the injection layer 18 may include one or more fractures (e.g., hydraulic fractures, natural fractures), fissures, and/or faults that enable the injection layer 18 to receive and store the CO₂. That is, the injection layer 18 and/or features thereof may define a reservoir 19 (e.g., CO₂ reservoir) configured to store CO₂ injected into the injection layer 18. The one or more sealing layers 20 may be positioned above and/or below (e.g., directly above, directly below, may overlay) the injection layer 18, thereby sealing the injection layer 18 (e.g., blocking CO₂ from traversing through the geological formation into the sealing layer(s) 20). For example, the one or more sealing layers 20 may include subsurface rock that has less than a threshold porosity and/or is impermeable, such that fluid (e.g., CO₂) is blocked from traversing through the sealing layer(s) 20. Thus, the one or more sealing layers 20 may be configured to limit the CO₂ injected into the injection layer 18 from reaching the one or more additional layers 22 and/or the atmosphere.

In the illustrated embodiment, the wellbore of the injection well 14 is completed with a casing 30 (e.g., cemented casing). For example, the casing 30 may extend along the injection well 14, such that the outer diameter of the casing 30 and the open hole 13 collectively define an annular space 15 through which cement may be pumped to seal the open hole 13 and the injection well 14. The cement may be configured to block carbon dioxide from traversing along the annular space 15 into the layers 20, 22 surrounding the injection layer 14 and/or into the atmosphere. That is, the cement may be pumped into the annular space 15 and may be configured to bond with the outer diameter of the casing 30 and with the open hole 13, such that the cement occupies the annular space 15, thereby limiting fluid flow (e.g., CO₂) through the annular space 15. In certain embodiments, the casing 30 and/or cement within the annular space 15 may be perforated at least in an interval 24 that intersects and/or aligns with the injection layer 18, thereby enabling CO₂ to be pumped and/or injected into the reservoir 19 of the injection layer 18. For example, in certain embodiments, a downhole tool 50 may be deployed into the injection well 14 and the downhole tool 50 may be located at a position corresponding to the intersection of the injection layer 18 with the injection well 14. Upon locating the downhole tool 50 within the interval 24, the downhole tool 50 may be operated to inject carbon dioxide through the perforations extending through the casing 30 and cement and into the reservoir 19 of the injection layer 18.

As noted above, it may be desirable to monitor the geological formation 12 to assess the integrity of the storage site (e.g., integrity of the injection layer 18, integrity of the reservoir 19) and/or to identify potential leaks of CO₂ into the various layers of the geological formation 12 (e.g., after CO₂ has been injection into the reservoir 19 via operation of the downhole tool 50). To this end, the CCSS 10 may include a monitoring system 40 having one or more monitoring lines 42 (e.g., fiber optic lines, electrical and/or optical telemetry cables, tubing-encased fiber optic line [TEF], tubing-encased cable [TEC], monitoring cable, other monitoring lines) that extend along an outer diameter of the casing 30 (e.g., extend along and through the annular space 15 defined by the casing 30 and the open hole 13). The one or more monitoring lines 42 may monitor temperature, acoustics, electromagnetic radiation, pressure, among other properties of the geological formation. A monitoring line 42 across a sealing layer (e.g., caprock) may monitor properties of the sealing layer. The one or more monitoring lines 42 may be configured to monitor the geological formation 12 for the presence of CO₂ within the annular space 15 and/or within the sealing layers 20 and/or additional layers 22 of the geological formation 12. In certain embodiments, at least a portion of the monitoring system 40 (e.g., portions of the monitoring lines 42) may be integrated, incorporated, and/or retained within the casing 30 to improve cementing conditions along the annulus 15, thereby limiting migration of carbon dioxide from the injection layer 18 into the sealing layer(s) 20 and/or additional layers 22 (e.g., via the annulus 15). For example, the casing 30 may include one or more sections and/or portions 32 configured to receive the one or more monitoring lines 42, such that the one or more monitoring lines 42 are at least partially retained within the casing 30.

In certain embodiments, each of the one or more sections 32 may include and/or be defined by a mandrel that circumferentially surrounds (e.g., circumscribes) the casing 30. The mandrel may be coupled to and/or integral with the casing 30. In certain embodiments, the mandrel may increase a diameter and/or circumference of the casing 30 (e.g., at least along the sections 32), thereby enabling the mandrel (e.g., the casing 30) to receive and/or retain the one or more monitoring lines 42. For example, the sections 32 defined by the mandrel(s) may at least partially define one or more cavities or passages extending along the length of the casing 30, whereby the one or more cavities or passages are configured to receive the one or more monitoring lines 42 of the monitoring system 40. In certain embodiments, the one or more cavities or passages may be further defined by additional components of the monitoring system 40, as discussed in greater detail below. For example, the monitoring system 40 may include various components that operate in conjunction with the casing 30 to retain the one or more monitoring lines 42 within the cavities and/or passages at least partially defined by the casing 30.

Further, in certain embodiments, the casing 30 may be designed and/or configured such that the one or more sections 32 of the casing 30 that at least partially define the passages and/or cavities configured to receive the monitoring lines 42 align with the one or more sealing layers 20 of the geological formation 12. In this way, cementing operations along the annular space 15 at a position corresponding to the sealing layers 20 may be improved, thereby improving the integrity of the storage site. It should be appreciated, however, that while the casing 30 is illustrated as having various sections 32 formed via one or more mandrels, where the sections 32 are configured to receive and/or retain the monitoring lines 42 and align with the one or more sealing layers 20, in certain embodiments, the sections 32 may extend for an entire length of the injection well 14. That is, while certain portions of the monitoring lines 42 are shown as extending along the casing 30 without being retained by the sections 32 of the casing 30, in other embodiments, the sections 32 may extend along an entire length of the injection well 14, such that the monitoring lines 42 are retained within the sections 32 along the entire length of the injection well 14. Further, it should be appreciated that while a single injection layer 18, two sealing layers 20, and three additional layers 22 are illustrated, the geological formation 12 may have any suitable number and/or combination of injection layers 18, sealing layers 20, and additional layers 22, so long as each of the injection layers 18 are surrounded by one or more sealing layers 20.

The monitoring system 40 discussed herein, which includes the casing 30, may be arranged and/or configured in a manner that facilitates proper cementing conditions, thereby improving the integrity of the storage site. For example, as noted above, in traditional systems, the monitoring lines may extend freely (e.g., unsecured) along the annular space 15 (e.g., extend along the annulus 15 without being integrated with the casing 30). However, the arrangement of unsecured monitoring lines along the annular space 15 may interfere with cement flow and/or may limit a bond (e.g., bonding seal) between the cement, the casing 30, and the open hole 13.

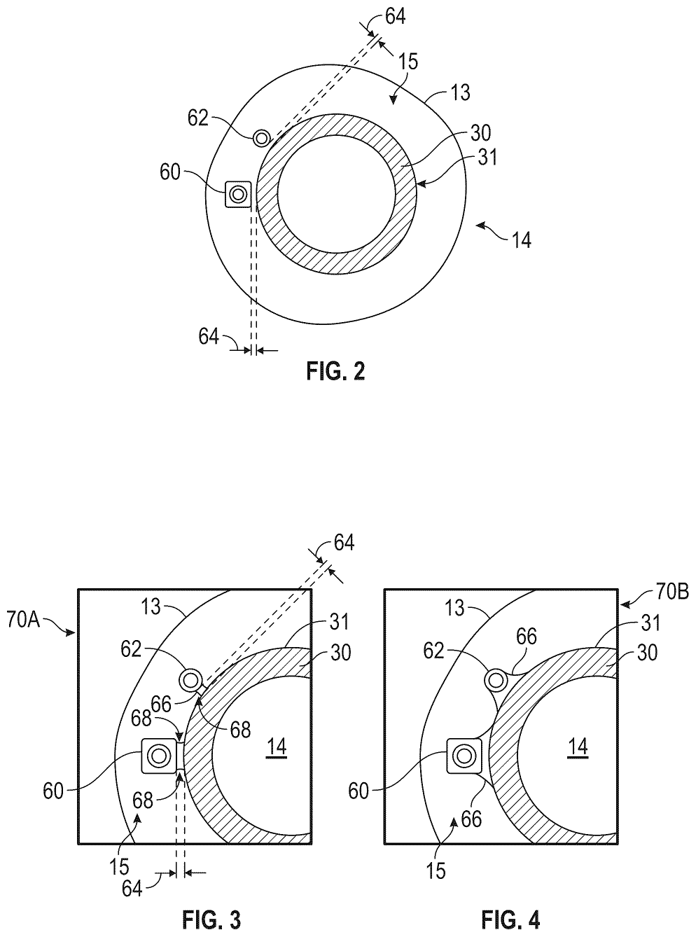

For example, FIG. 2 is a cross-sectional view of an embodiment of an injection well 14 having a casing 30 with an outer diameter 31, where the casing 30 and the open hole 13 collectively define an annular space 15 in which cement may be pumped to seal the annular space 15. As shown in the illustrated embodiment, one or more monitoring lines (e.g., unsecured monitoring lines) extend through the annular space 15. For example, an encapsulated monitoring line 60 and a bare monitoring line 62 extend through the annular space 15, where each of the encapsulated monitoring line 60 and bare monitoring line 62 are unsecured to the casing 30 (e.g., not integrated and/or incorporated with the casing 30). In certain embodiments, the encapsulated monitoring line 60 may correspond to a monitoring line that includes a protective structure (e.g., tubing) circumscribing the monitoring line. For example, the encapsulated monitoring line 60 may be a tubing encased fiber (TEF) optic line or a tubing encased cable (TEC) line. In certain embodiments, when the monitoring lines 60, 62 are unsecured to the casing 30, a gap 64 (e.g., radial gap, small space) may exist and/or be formed between the monitoring line(s) 60, 62 and the outer diameter 31 of the casing 30. In certain cases, a size of the gap 64 (e.g., small gap) may limit and/or block cement from flowing between the monitoring lines 60, 62 and the outer diameter 31 of the casing 30, thereby preventing a cement bond from being formed between the casing 30 and the cement (e.g., at least in the interval of the gap 64). That is, because a limited amount of space (e.g., less than a threshold amount of space, less than 0.5 inches, less than 1.27 centimeters) is provided within the gap 64, cement may not flow readily (e.g., at a threshold mass flow rate) within and/or through the gaps 64. As a result, a micro-annulus may be formed within the annular space 15 between the outer diameter 31 of the casing 30 and the monitoring lines 60, 62 (e.g., along the gap 64), and the micro-annulus may be susceptible to carbon dioxide leakage.

Additionally, or alternatively, the arrangement of unsecured monitoring lines 60, 62 in traditional systems may cause the monitoring lines 60, 62 to extend along the annular space 15 in an unpredictable manner, thereby causing variations in radial and/or circumferential positions of the monitoring lines 60, 62. For example, because the monitoring lines 60, 62 are unsecured (e.g., extend freely about the annular space 15), as cement is injected through the annular space 15 and around the monitoring lines 60, 62, the monitoring lines 60, 62 may move radially and/or circumferentially about the annular space 15. Such movement may obstruct the flow of cement through the annular space 15 and/or limit the cement from forming a secure bond with the monitoring lines 60, 62, the casing 30, and/or the open hole 15, which may lead to the formation of additional micro-annuluses. In certain cases, the unsecured monitoring lines 60, 62 may also cause a bonding surface for the cement to be uneven or non-uniform, thereby further reducing the efficacy and/or efficiency of cementing operations. For example, cement may have a greater tendency to readily bond (e.g., form a secure bond) with uniform (e.g., flat, smooth) surfaces relative to non-uniform surfaces (e.g., jagged surfaces, surface with bends or irregularities). Thus, arrangements that include unsecured monitoring lines 60, 62 may be associated with a greater tendency or likelihood of improper cementing based on the non-uniform surfaces provided by the unsecured monitoring lines 60, 62.

In certain cases, an adhesive may be used to secure the monitoring lines 60, 62 to the outer diameter 31 of the casing 30. For example, FIGS. 3 and 4 illustrate a first monitoring system 70A and a second monitoring system 70B, respectively, that utilize an adhesive 66 (e.g., glue) to secure the monitoring lines 60, 62 to the outer diameter 31 of the casing 30. In certain embodiments, the adhesive 66 may be positioned between the monitoring lines 60, 62 and the outer diameter 31 of the casing 30 (e.g., within the gaps 64) to couple (e.g., secure) the monitoring lines 60, 62 to the casing 30. While the adhesive 66 may improve cementing conditions along the annular space 15 relative to systems that do not employ an adhesive, arrangements using the adhesive 66 may still be susceptible to improper cementing. For example, as shown in FIG. 3, application of the adhesive 66 between the monitoring lines 60, 62 and the outer diameter 31 of the casing 30 may cause the formation of a micro-annulus at a crevice 68 between the adhesive 66 and the casing 30, which may be undesirable. In certain embodiments, the adhesive 66 may be applied, such that a smooth transition is provided between the monitoring lines 60, 62 and the casing 30 via the adhesive. For example, as shown in FIG. 4, the adhesive 66 may be applied such that the adhesive 66 extends tangentially from a surface of the monitoring lines 60, 62 and tangentially from the outer diameter 31 of the casing 30. In this way, formation of crevices 68 may be reduced, thereby improving cementing conditions. However, application of the adhesive 66 to the casing 30 and the monitoring lines 60, 62 such that a smooth transition is provided (e.g., such that the adhesive extends tangentially from the outer diameter 31 of the casing 30 and tangentially from the monitoring lines 60, 62) may be difficult to achieve consistently.

Returning to FIG. 1, by integrating the monitoring lines 42 of the monitoring system 40 with the casing 30, cementing conditions along the injection well 14 may be improved. For example, integrating the monitoring lines 42 of the monitoring system 40 with the casing 30 may eliminate the gaps 64 discussed above, thereby increasing the amount of space within the annular space 15, such that cement flow may be improved. Further, by eliminating the gaps 64, the formation of micro-annuluses along the annular space 15 may be reduced, thereby further improving the efficacy of cementing operations performed along the annular space 15 and/or improving the integrity of the storage site. Additionally, or alternatively, integrating the monitoring lines 42 of the monitoring system 40 with the casing 30 may provide a continuous or uniform surface (e.g., smooth surface) that is capable of forming a secure bond with cement introduced into the annular space 15. For example, portions and/or components of the monitoring systems discussed herein that further define the cavities and/or passages at least partially defined by the casing 30 may be configured to provide a continuous surface (e.g., uniform surface, smooth surface) that is flush with the outer diameter 31 of the casing 30, as discussed in greater detail below. The continuous surface may improve the quality of a cement bond formed between the casing 30 and the cement, thereby improving cementing operations and/or increasing the integrity of the storage site.

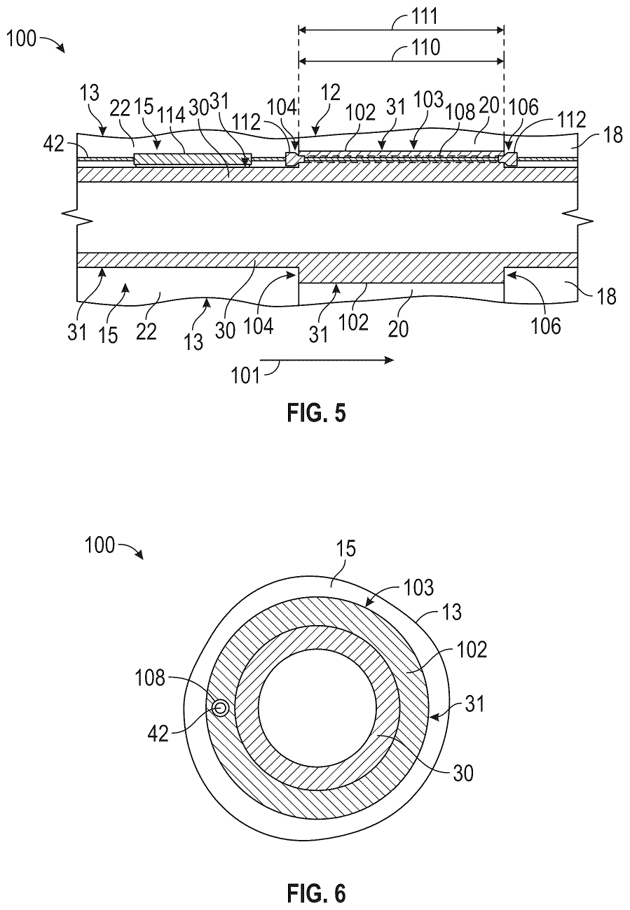

FIGS. 5 and 6 illustrate an embodiment of a monitoring system 100 (e.g., monitoring system 40) that may be employed by the CCSS 10 to monitor and/or assess the integrity of a storage site (e.g., assess the integrity of the layers 18, 20, 22 of the geological formation 12). For example, FIG. 5 is a schematic view of the monitoring system 100, and FIG. 6 is a cross-sectional view of the monitoring system 100. The monitoring system 100 may include similar features to the monitoring system 40 of FIG. 1 and/or may correspond to the monitoring system 40 of FIG. 1. For example, the monitoring system 100 may include the monitoring lines 42 that may be integrated with and/or incorporated into the casing 30 of the injection well 14. That is, portions of the monitoring system 100 may be at least be partially defined by the casing 30 (e.g., sections 32 of the casing 30), thereby enabling the monitoring system 100 to monitor and/or assess the integrity of the storage site while providing for substantially improved or optimal cementing conditions, as discussed above.

As shown in FIG. 5, the casing 30 includes a mandrel 102 (e.g., annular mandrel) having a first end 104 (e.g., upstream end relative to a flow direction 101 of CO₂ into the geological formation 12) and a second end 106 (e.g., downstream end relative to the flow direction 101 of CO₂ into the geological formation). The mandrel 102 may be configured to define a passage 108 that extends along a length 110 of the mandrel 102 from the first end 104 to the second end 106. The passage 108 may be configured to receive and/or retain the monitoring line(s) 42 of the monitoring system 100. For example, the mandrel 102 may be integral with and/or coupled to the casing 30 (e.g., annular casing), such that an outer surface 103 (e.g., outer annular surface) of the mandrel 102 forms the outer diameter 31 of the casing 30 (e.g., at least along the length 110 of the mandrel 102). That is, the mandrel 102 may encapsulate a portion of the casing 30 and may be configured to increase a diameter of a portion of the casing 30 such that the passage 108 may be formed along the casing 30. In certain embodiments, a size (e.g., diameter, circumference) of the passage 108 defined by the mandrel 102 may be selected based on a size (e.g., diameter, circumference) of the monitoring line(s) 42. For example, a size of the passage 108 may be larger than a size of the monitoring line(s) 42, thereby enabling the monitoring line(s) 42 to extend through the passage 108. Further, the mandrel 102 may be concentric to the open hole 13 (e.g., as shown in FIG. 6) to provide sufficient space for proper cementing, as discussed below.

In certain embodiments, the mandrel 102 may correspond to the sections 32 of the casing 30 illustrated in FIG. 1. For example, in certain embodiments, the mandrel 102 may be configured to align with the sealing layers 20 of the geological formation 12, such that the monitoring line 42 is integrated within the casing 30 for a distance that extends along the sealing layers 20. In certain embodiments, the length 110 of the mandrel 102 (e.g., from the first end 104 to the second end 106) may be selected and/or designed based on a dimension 111 (e.g., length, depth) of the sealing layers 20. For example, for sealing layers 20 that extend for a depth of ten meters, the length 110 of the mandrel 102 may also extend ten meters, such that the monitoring line(s) 42 of the monitoring system 100 extending through the passage 108 defined by the mandrel 102 are contained within and/or integrated with the casing 30 along the entire depth of the sealing layers 20. That is, the length 110 of the mandrel 102 may be substantially similar to (e.g., equal to) the length or depth 111 of the sealing layers 20, thereby providing substantially improved or optimal cementing conditions in locations that correspond to the sealing layers 20.

For example, by employing the mandrel 102 that defines the passage 108 configured to receive the monitoring lines 42, the monitoring lines 42 may be secured within the passage 108, thereby eliminating potential gaps (e.g., gaps 64) between the monitoring lines 42 and the outer diameter 31 of the casing 30. Further, by securing the monitoring lines 42 within the casing 30 (e.g., within the mandrel 102), additional space between the outer surface 103 of the mandrel 102 and the open hole 13 may be provided that enables cement to readily flow therethrough to seal the annular space 15. The outer surface 103 of the mandrel 102 may also provide a continuous (e.g., uniform, smooth) surface, thereby improving the quality of a cement bond formed between the cement and the outer surface 103 of the mandrel 102. Further still, with the monitoring lines 42 secured within the mandrel 102, the monitoring lines 42 may be limited from moving radially and/or circumferentially about the annular space 15, such that the monitoring lines do not interfere with the flow of cement through the annular space 15, as discussed above. That is, by integrating the monitoring lines 42 within the casing 30, the monitoring lines 42 may extend through the annular space in a predictable manner, thereby reducing bends and/or turns in the monitoring line 42 that may otherwise affect or hinder cementing operations.

In certain embodiments, the mandrel 102 may include a fitting 112 (e.g., annular fitting and/or bushing) disposed at the first end 104 and/or second end 106 of the mandrel 102. The fitting(s) 112 may be configured to centralize a position of the monitoring line(s) 42 within the passage 108 and/or seal the passage 108 after the monitoring line(s) 42 are directed through the passage 108. In certain embodiments, the fitting(s) 112 may correspond to a hydraulic dry-mate connector (HDMC) fitting or an instrumentation double ferrule compression (IDFC) fitting. Further, the monitoring system 100 may include a splicing device 114 configured to rejoin portions of the monitoring line(s) 42 that have been cut during installation of the monitoring line(s) 42 into the passages 108. For example, in order to direct the monitoring line(s) 42 through the passage 108, the monitoring line(s) 42 may be cut in various locations. The splicing device 114 may be configured to recouple portions of the monitoring line(s) 42 to one another such that the monitoring line(s) 42 extend along the length of the casing 30.

It should be appreciated that while FIG. 5 illustrates and/or describes the mandrel 102 as extending for a length (e.g., axial length) that is substantially similar to a dimension (e.g., depth 111) of the sealing layer(s) 20, in other embodiments, the mandrel 102 may extend for any length of the casing 30. For example, the mandrel 102 may extend along an entire length of the casing 30 and/or along a length of the casing that aligns with multiple different layers (e.g., injection layer 18, additional layers 22). That is, in certain embodiments, the mandrel 102 (e.g., sections 32) may extend beyond the sealing layers 20, such that the passage 108 defined by the mandrel 102 also extends beyond the sealing layers 20. In embodiments in which the mandrel 102 extends along an entire length of the casing 30, the fittings 112 and/or the splicing device 114 may be omitted. Further, while a single passage 108 is illustrated in FIGS. 5 and 6, it should be appreciated that the mandrel 102 may include any number (e.g., two, three, four, or more) of passages 108 extending therethrough to receive any number (e.g., one, two, three, four, or more) of monitoring lines 42. That is, the mandrel 102 may include a single passage 108 configured to receive any number (e.g., one, two, three, or more) of monitoring lines 42, or the mandrel 102 may include any number of passages 108, where each passage 108 is capable of receiving any number (e.g., one, two, three, or more) of monitoring lines 42.

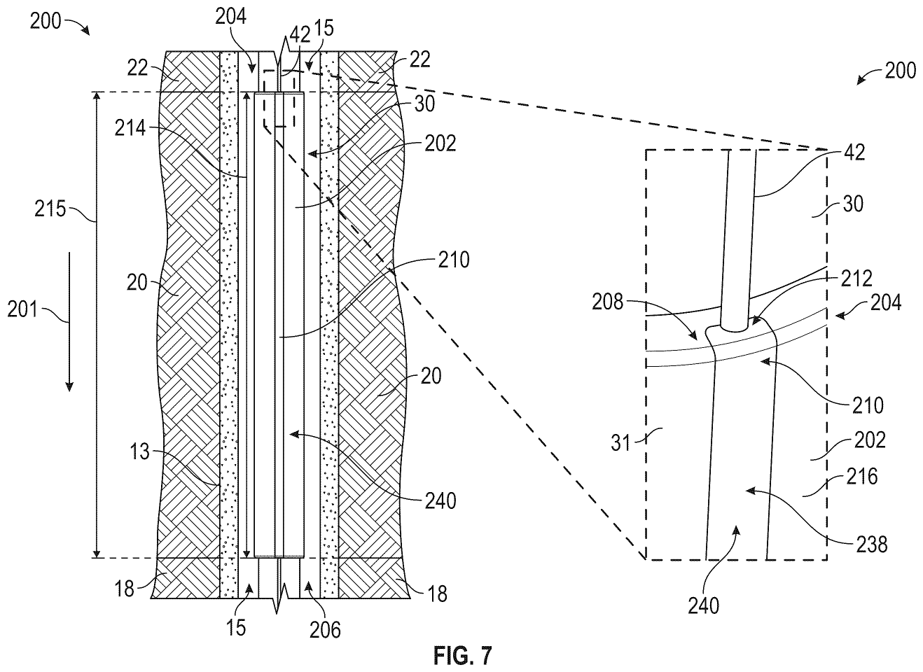

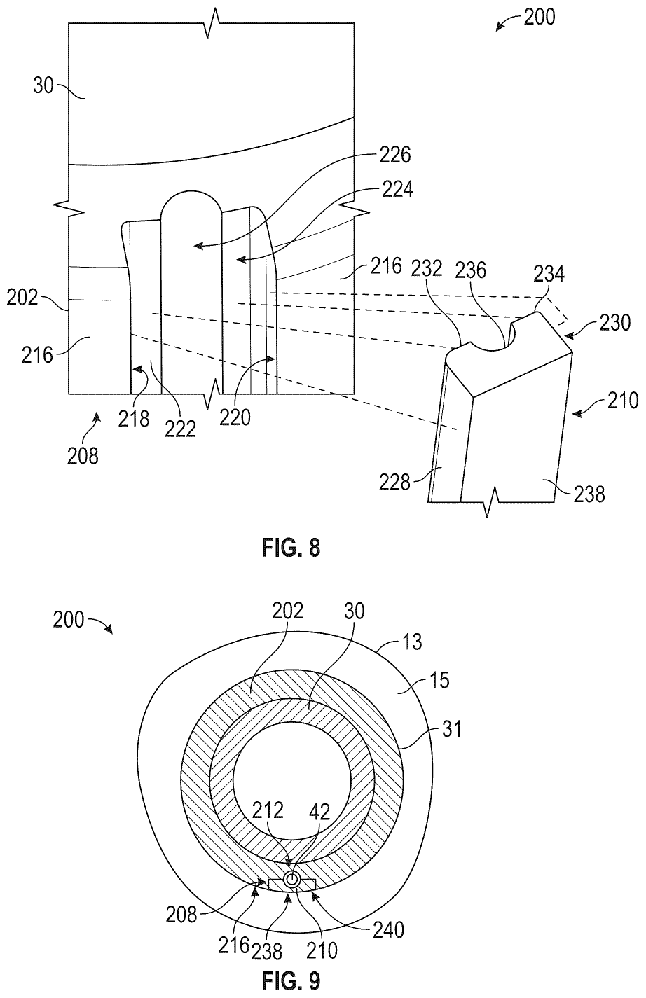

FIGS. 7-9 illustrate an embodiment of a monitoring system 200 (e.g., monitoring system 40) and/or components thereof that may employed by the CCSS 10 to monitor and/or assess the integrity of a storage site (e.g., assess the integrity of the layers 18, 20, 22 of the geological formation 12). For example, FIG. 7 is a perspective view of an embodiment of the monitoring system 200 in an assembled configuration, FIG. 8 is an exploded perspective view of an embodiment of the monitoring system 200, and FIG. 9 is a cross-sectional view of an embodiment of the monitoring system 200 in an assembled configuration. The monitoring system 200 may include similar features to the monitoring system 40 of FIG. 1 and/or may correspond to the monitoring system 40 of FIG. 1. For example, the monitoring system 200 may include the monitoring lines 42 that may be integrated and/or incorporated within the casing 30 of the injection well 14. That is, portions of the monitoring system 200 may be at least partially defined by the casing 30 (e.g., sections 32 of the casing 30), thereby enabling the monitoring system 200 to monitor and/or assess the integrity of the storage site while providing for substantially improved or optimal cementing conditions, as discussed above.

As shown in FIG. 7, the casing 30 includes a mandrel 202 (e.g., annular mandrel) having a first end 204 (e.g., upstream end relative to a flow direction 201 of CO₂ into the geological formation 12) and a second end 206 (e.g., downstream end relative to the flow direction 201 of CO₂ into the geological formation 12). The mandrel 202 may include a recess 208 (e.g., axial slot, groove, or recessed portion) configured to receive one or more binding agents 210 (e.g., axial inserts) of the monitoring system 200, where the recess 208 and the insert(s) 210 cooperatively define a cavity 212 (e.g., axial passage) that extends axially along a length 214 of the mandrel 202. In certain embodiments, the insert(s) 210 may be plastic inserts, whereas in other embodiments, the insert(s) 210 may be metal inserts or elastomer inserts.

As shown in FIG. 8, the mandrel 202 includes an outer surface 216 (e.g., outer annular surface, outer diameter), and the recess 208 may extend radially inward (e.g., relative to a central axis of the injection well 14) from the outer surface 216, thereby enabling the recess 208 to receive the insert(s) 210. That is, the recess 208 may include a first surface 218 (e.g., first radial surface) extending in a direction (e.g., radial direction, radially inward direction) from the outer surface 216 toward an inner surface (e.g., inner diameter) of the casing 30, a second surface 220 (e.g., second radial surface) extending in a direction (e.g., radial direction, radially inward direction) from the outer surface 216 toward the inner surface (e.g., inner diameter) of the casing 30, a third surface 222 (e.g., first circumferential surface) extending in a direction (e.g., circumferential direction, crosswise direction relative to the first surface 218) from the first surface 218, a fourth surface 224 (e.g., second circumferential surface) extending in a direction (e.g., circumferential direction, crosswise direction relative to the second surface 220) from the second surface 220, and fifth surface 226 (e.g., arcuate surface, curved surface, U-shaped surface, or semi-cylindrical surface) extending between the third surface 222 and the fourth surface 224. Each of the surfaces 218, 220, 222, and 224 may be configured to engage with (e.g., abut) surfaces of the insert(s) 210, and the fifth surface 226 may at least partially define the cavity 212, thereby enabling the monitoring line 42 to be retained within the cavity 212. That is, a geometry (e.g., shape) of the insert(s) 210 may be based on a geometry (e.g., shape) of the recess 208 and the monitoring line(s) 42, thereby enabling the monitoring line(s) 42 to be retained within the cavity 212.

For example, the insert(s) 210 may include a first surface 228 (e.g., first radial surface) extending in a direction (e.g., radial direction) in an assembled configuration of the monitoring system 200, a second surface 230 (e.g., second radial surface) extending in a direction (e.g., radial direction) in an assembled configuration of the monitoring system 200, a third surface 232 (e.g., first circumferential surface) extending in a direction (e.g., circumferential direction, crosswise direction) from the first surface 228, a fourth surface 234 (e.g., second circumferential surface) extending in a direction (e.g., circumferential direction, crosswise direction) from the second surface 230, and a fifth surface 236 (e.g., arcuate surface, curved surface, U-shaped surface, or semi-cylindrical surface) extending between the third surface 232 and the fourth surface 234.

The first surface 228 of the insert(s) 210 may be configured to engage with and/or abut the first surface 218 of the recess 208, the second surface 230 of the insert(s) 210 may be configured to engage with and/or abut the second surface 220 of the recess 208, the third surface 232 of the insert(s) 210 may be configured to engage with and/or abut the third surface 222 of the recess 208, and the fourth surface 234 of the insert(s) 210 may be configured to engage with and/or abut the fourth surface 224 of the recess 208. Thus, upon assembly of the insert(s) 210 into the recess 208, the fifth surface 226 of the recess 208 and the fifth surface 236 of the insert(s) 210 may cooperatively define the cavity 212 (e.g., cylindrical passage) configured to receive and/or retain (e.g., capture) the monitoring line(s) 42. For example, during assembly of the monitoring system 200, the monitoring line 42 may be inserted and/or placed into the recess 208 (e.g., against the fifth surface 226 of the recess 208). Thereafter, the insert 210 may be placed within the recess 208, such that the fifth surface 236 of the insert 210 engages with the monitoring line 42, thereby enabling the monitoring line(s) 42 to be held, retained, and/or centralized within the cavity 212 defined by the fifth surface 226 of the recess 208 and the fifth surface 236 of the insert(s) 210. Additionally, the insert(s) 210 may include a sixth surface 238 (e.g., exterior surface) configured to align with the outer surface 216 of the mandrel 202 in an assembled configuration of the monitoring assembly 200. For example, upon assembly of the insert(s) 210 into the recess 208, the sixth surface 238 of the insert(s) 210 and the outer surface 216 of the mandrel 202 may be flush with one another, thereby providing a continuous, uniform, and/or smooth surface 240 (shown in FIG. 7) for cementing operations. That is, the insert 210 and the outer surface 216 of the mandrel 202 may enable the cement to more completely fill the space between the open hole and the casing without interference by the monitoring line(s) 42, thereby reducing or eliminating leakage paths for CO₂ across the mandrel 202. In certain embodiments, the insert(s) 210 may be retained within the recess 208 via an adhesive. For example, an adhesive may be applied to one or more of the surfaces 218, 220, 222, 224, 228, 230, 232, and 234, thereby enabling the surfaces of the recess 208 and the insert(s) to couple to one another to secure the insert(s) 210 within the recess 208. In certain embodiments, the insert(s) 210 may be retained within the recess 208 via a snap fit or an interference fit. In certain embodiments, the insert(s) 210 (e.g., metal insert(s)) may be retained within the recess 208 via a welded joint, a brazed joint, or any combination thereof.

Returning to FIG. 7, the mandrel 202 may be integral with and/or coupled to the casing 30, such that the outer surface 216 (e.g., outer annular surface) of the mandrel 202 forms the outer diameter 31 of the casing 30 (e.g., at least along the length 214 of the mandrel 202). That is, the mandrel 202 may encapsulate a portion of the casing 30 and may be configured to increase a diameter of a portion of the casing 30, such that the cavity 212 may be formed along the casing 30. In certain embodiments, a size (e.g., diameter, circumference) of the cavity 212 may be designed and/or selected based on a size (e.g., diameter, circumference) of the monitoring line(s) 42 extending therethrough. For example, in certain embodiments, a size of the cavity 212 may be larger than a size of the monitoring line(s) 42, thereby enabling the monitoring line(s) 42 to extend through the cavity 212. Further, the mandrel 202 may be concentric to the open hole 13 (e.g., as shown in FIG. 9) to provide sufficient space for proper cementing, as discussed below.

In certain embodiments, the mandrel 202 may correspond to the sections 32 of the casing 30 illustrated in FIG. 1. For example, in certain embodiments, the mandrel 202 may be configured to align with the sealing layers 20 of the geological formation 12, such that the monitoring line 42 is integrated within the casing 30 for a distance that extends along the sealing layers 20. In certain embodiments, the length 214 of the mandrel 202 (e.g., from the first end 204 to the second end 206) may be selected and/or designed based on a dimension 215 (e.g., length, depth) of the sealing layers 20. For example, for sealing layers 20 that extend for a depth of ten meters, the length 214 of the mandrel 202 may also extend ten meters, such that the monitoring line(s) 42 of the monitoring system 200 extending through the cavity 212 defined by the recess 208 of mandrel 202 and the insert(s) 210 (e.g., defined by the fifth surface 226 of the recess 208 and the fifth surface 236 of the insert(s) 210) are contained within and/or integrated with the casing 30 along the entire depth of the sealing layers 20. That is, the length 214 of the mandrel 102 may be substantially similar to the length or depth 215 of the sealing layers 20, thereby providing substantially improved or optimal cementing conditions in locations that correspond to the sealing layers 20. In certain embodiments, the insert 210 may be a single piece that extends for the length 214 of the mandrel 202, while in other embodiments, multiple inserts 210 may be employed such that a sum of the respective lengths of the multiple inserts 210 is equal to the length 214 of the mandrel 202.

By employing the monitoring system 200 (e.g., the mandrel 202 having the recess 208 and the insert(s) 210) that defines the cavity 212 configured to receive the monitoring lines 42, the monitoring lines 42 may be secured within the cavity 212, thereby eliminating potential gaps (e.g., gaps 64) between the monitoring lines 42 and the outer diameter 31 of the casing 30. Further, by securing the monitoring lines 42 within the casing 30 (e.g., within the mandrel 202), additional space within the annular space 15 (e.g., between the outer surface 216 of the mandrel 202 and the open hole 13) may be provided that enables cement to readily flow therethrough to seal the annular space 15. The outer surface 216 of the mandrel 202 along with the sixth surface 238 of the insert(s) 210 may also provide a continuous (e.g., uniform, smooth, flush) surface (e.g., surface 240), thereby improving the quality of a cement bond formed between the cement and the surface 240 of the mandrel 202. Further still, with the monitoring lines 42 secured within the mandrel 202, the monitoring lines 42 may be limited from moving about the annular space 15, such that an amount of interference with the flow of cement through the annular space 15 (e.g., via the monitoring lines 42) is reduced or limited, as discussed above. That is, by integrating the monitoring lines 42 within the casing 30, the monitoring lines 42 may extend through the annular space 15 in a predictable manner, thereby reducing bends and/or turns in the monitoring line 42 that may otherwise affect or hinder cementing operations.

It should be appreciated that while FIG. 7 illustrates and/or describes the mandrel 202 as extending for a length 214 that is substantially similar to a dimension of the sealing layer(s) 20, in other embodiments, the mandrel 202 (and thus the recess 208 and insert(s) 210) may extend for any length of the casing 30. For example, the mandrel 202 may extend along an entire length of the casing 30 and/or along a length of the casing that aligns with multiple different layers (e.g., injection layer 18, additional layers 22). That is, in certain embodiments, the mandrel 202 (e.g., sections 32) may extend beyond the sealing layers 20, such that the cavity 212 defined by the mandrel 202 and the insert(s) 210 also extends beyond the sealing layers 20. Further, while a single cavity 212 is illustrated in FIGS. 7-9, it should be appreciated that the mandrel 202 may include any number (e.g., two, three, four, or more) of cavities 212 (e.g., defined by recesses 208 and insert(s) 210) extending therethrough to receive any number (e.g., one, two, three, four, or more) of monitoring lines 42. That is, the mandrel 202 may include a single cavity 212 configured to receive any number (e.g., one, two, three, or more) of monitoring lines 42, or the mandrel 202 may include any number of cavities 212, where each cavity 212 is capable of receiving any number (e.g., one, two, three, or more) of monitoring lines 42.

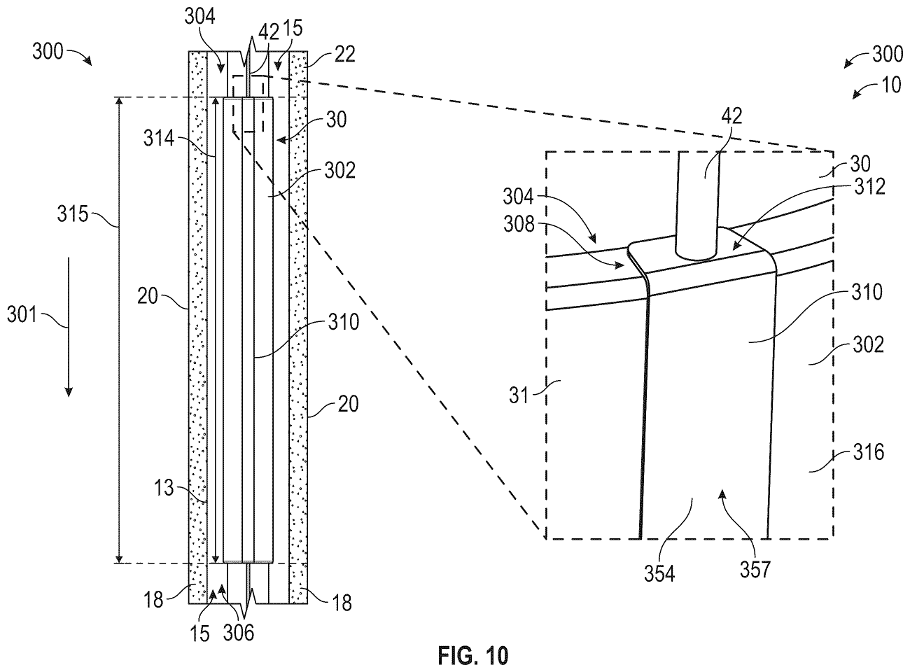

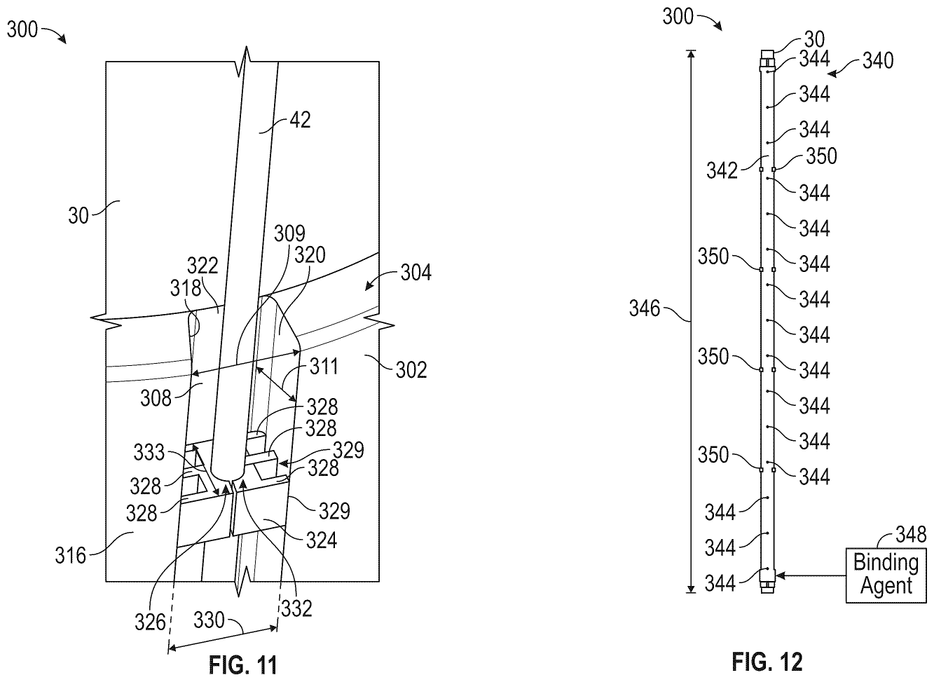

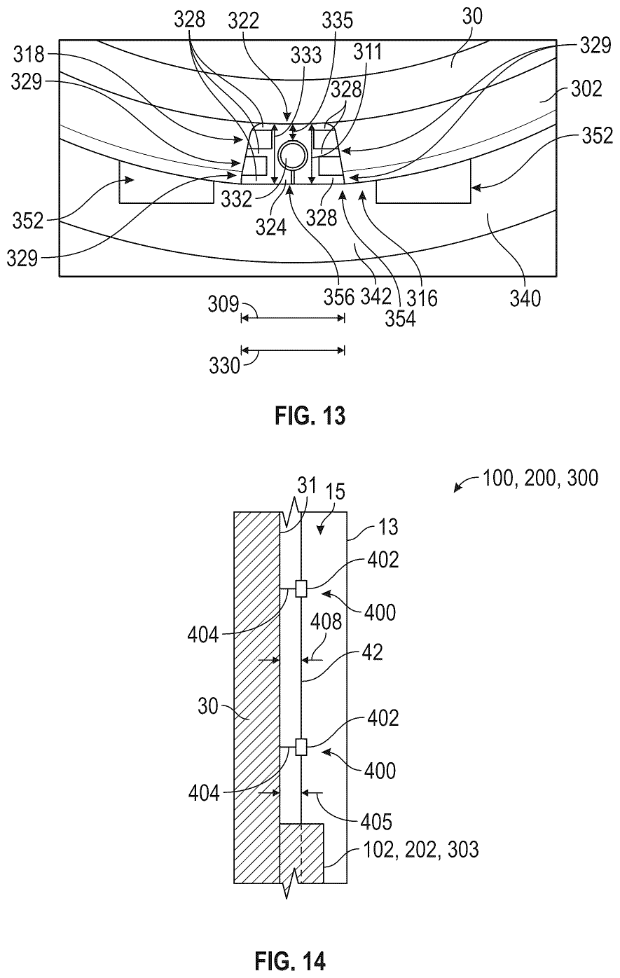

FIGS. 10-13 illustrate an embodiment of a monitoring system 300 (e.g., monitoring system 40) that may employed by the CCSS 10 to monitor and/or assess the integrity of a storage site (e.g., assess the integrity of the layers 18, 20, 22 of the geological formation 12). For example, FIG. 10 is a perspective view of an embodiment of the monitoring system 300 in an assembled configuration, FIG. 11 is a perspective view of an embodiment of the monitoring system 300, FIG. 12 is a perspective view of an embodiment of a casing strap of the monitoring system 300, and FIG. 13 is a cross-sectional view of an embodiment of the monitoring system 300. The monitoring system 300 may include similar features to the monitoring system 40 of FIG. 1 and/or may correspond to the monitoring system 40 of FIG. 1. For example, the monitoring system 300 may include the monitoring lines 42 that may be integrated and/or incorporated within the casing 30 of the injection well 14. That is, portions of the monitoring system 300 may at least partially defined by the casing 30 (e.g., sections 32 of the casing 30), thereby enabling the monitoring system 300 to monitor and/or assess the integrity of the storage site while providing for substantially improved or optimal cementing conditions, as discussed above.

As shown in FIG. 10, the casing 30 includes a mandrel 302 having a first end 304 (e.g., upstream end relative to a flow direction 301 of CO₂ into the geological formation 12) and a second end 306 (e.g., downstream end relative to the flow direction 301 of CO₂ into the geological formation 12). The mandrel 302 may include a recess 308 (e.g., axial slot, groove, or recessed portion) configured to receive a binding agent 310 (e.g., curable binding agent, injectable binding agent, epoxy, specialized cement) of the monitoring system 300, where the recess 308 and the binding agent 310 cooperatively define a cavity 312 (e.g., axial passage) that extends axially along a length 314 of the mandrel 302. In certain embodiments, the binding agent 310 may correspond to an epoxy (e.g., cured epoxy) or a specialized cement configured to cure or harden within the recess 308 (e.g., after a threshold amount of time) to form the cavity 312 through which the monitoring lines 42 extend therethrough.

For example, FIG. 11 illustrates the recess 308 of the mandrel 302. In the illustrated embodiment, the mandrel 302 includes an outer surface 316 (e.g., outer annular surface, outer diameter), and the recess 308 may extend radially inward (e.g., relative to a central axis of the injection well 14) from the outer surface 316, thereby enabling the recess 308 to receive the binding agent 310. That is, the recess 308 may include a first surface 318 (e.g., first radial surface) extending in a direction (e.g., radial direction, radially inward direction) from the outer surface 316 toward an inner surface (e.g., inner diameter) of the casing 30, a second surface 320 (e.g., second radial surface) extending in a direction (e.g., radial direction, radially inward direction) from the outer surface 316 toward the inner surface (e.g., inner diameter) of the casing 30, and a third surface 322 extending in a direction (e.g., circumferential direction, crosswise direction relative to the surfaces 318, 320) between the first surface 318 and the second surface 320. Each of the surfaces 318, 320, 322 may be configured to engage with (e.g., couple to, bond with) the binding agent 310 to secure the monitoring line(s) 42 within the recess 308, as described in greater detail below. In certain embodiments, one or more centralizers 324 may be positioned within the recess 308 (e.g., prior to injection of the binding agent 310), and the centralizers 324 may be configured to retain and/or hold the monitoring line(s) 42 within a central portion of the recess 308 to provide sufficient space for the binding agent 310 to surround (e.g., circumscribe) the monitoring line to secure the monitoring line(s) 42 within the recess 308.

For example, the centralizer 324 may include a body 326 and one or more protrusions 328 extending in a direction (e.g., circumferential direction relative to the mandrel 302) from the body 326 in an assembled configuration of the monitoring system 300. In certain embodiments, ends 329 (e.g., distal ends) of the protrusions 326 may be configured to engage with the first and second surfaces 318, 320 to secure the centralizer 324 within the recess 308. For example, the recess 308 may have a width 309 and a width 330 of the centralizer 324 (e.g., a dimension of the centralizer 324 from an end 329 of a first protrusion 328, through the body 326, and to an end 329 of a second protrusion 328) may be substantially similar to the width 309 of the recess 308, thereby enabling the centralizer 324 to be secured within the recess 308. Additionally, the body 326 of the centralizer 324 may define a port 332 through which the monitoring line(s) 42 may extend. As noted above, in certain embodiments, the centralizer 324 may be configured such that the port 332 is centralized within the recess 308, thereby enabling the monitoring line(s) 42 to be centralized within the recess 308. For example, the recess 308 may have a depth 311, and a depth 333 of the centralizer 326 (e.g., a dimension of the centralizer 324 extending in a radial direction) may be substantially similar to the depth 311 of the recess 308. As shown in FIG. 13, the port 332 may be positioned at a midpoint along the width 330 and/or the depth 333 of the centralizer 324, such that the port 332 is centralized within the recess 308. Thus, in certain embodiments, the centralizer 324 may be configured to space the monitoring line 42 extending therethrough (e.g., extending through the port 332) at a distance 335 from the third surface 322 of the recess 308, such that a gap exists between the monitoring line 42 and the third surface 322 of the recess 308 (e.g., along portions of the monitoring line 42 that are not retained within a centralizer 324). In this way, the binding agent 310 may be injected into the recess 308 and may flow around the centralizer(s) 324 and the monitoring line(s) 42 (e.g., through the gap between the monitoring line 42 and the third surface 322 of the recess 308) to secure the monitoring line(s) 42 within the recess 308.

FIG. 12 illustrates a casing strap 340 configured to bias the binding agent 310 into the recess 308 to secure the monitoring line(s) 42 within the recess 308. During assembly of the monitoring system 300, the monitoring line(s) 42 may be passed through the centralizers 324, the centralizers 324 and the monitoring line(s) 42 may be inserted into the recess 308, and the casing strap 340 may be disposed around the mandrel 302. In this way, the casing strap 340 may facilitate injection of the binding agent 310 into the recess 308. For example, the casing strap 340 may include a body 342 having a number of ports 344 distributed along a length 346 of the casing strap 340. The casing strap 340 may be configured to receive the binding agent 310 from a binding agent source 348 (e.g., pump), and the casing strap 340 may be configured to distribute the binding agent 310 into the recess 308 along the length 346 of the casing strap 340 via the ports 344. That is, the binding agent source 348 may be configured to deliver the binding agent 310 under pressure to the casing strap 340, and the casing strap 340 may then be configured to inject the binding agent 310 through the ports 344 and into the recess 308 to secure the centralizer(s) 324 and the monitoring line(s) 42 within the recess 308.

In certain embodiments, the casing strap 340 may be configured to circumscribe the mandrel 302. For example, the body 342 of the casing strap 340 may at least partially circumscribe the mandrel 302, and the casing strap 340 may also include one or more collars 350 configured to surround the mandrel 302 to maintain a position of the casing strap 340 with the mandrel 302. During installation of the monitoring system 300, the body 342 of the casing strap 340 may be configured to align with the recess 308 of the mandrel 302 such that the ports 344 distributed along the body 342 also align with the recess 308 of the mandrel. In this way, the binding agent 310 may be injected into the recess 308 to secure the monitoring lines within the mandrel 302. Additionally, in certain embodiments, the casing strap 340 may include additional components to facilitate delivery of the binding agent 310 into the recess 308.

For example, as shown in FIG. 13, the casing strap 340 may include one or more seals 352 extending along the length 346 of the casing strap 340. The one or more seals 352 may be positioned on opposite sides of the recess 308 (e.g., within a threshold distance of the recess 308) and may be configured to limit migration of the binding agent 310 from the recess 308 during application of the binding agent 310 (e.g., during operation of the binding agent source 344 that delivers the binding agent 310 through the casing strap 340 and into the recess 308). During assembly of the monitoring system 300, the casing strap 340 may be configured to circumscribe the mandrel 302 for a threshold amount of time that corresponds to an amount of time for the binding agent 310 to cure within the recess 308. Thereafter, the casing strap 340 may be removed from the mandrel 302. Notably, by employing the casing strap 340, an exterior surface 354 (e.g., outer surface) of the binding agent 310 may be flush with the outer surface 316 of the mandrel 302. For example, an inner surface 356 of the casing strap 340 may align with the outer surface 354 of the mandrel 302, such that when the binding agent 310 is injected into the recess 308 via the casing strap 340, the binding agent 310 may impinge against the inner surface 356 of the casing strap 340. In this way, the outer surface 354 of the binding agent 310 may be made flush with the outer surface 316 of the mandrel 302 to form a continuous surface 357 (e.g., smooth surface, continuous surface, shown in FIG. 10), thereby improving cementing operations along the mandrel 302.

Returning to FIG. 10, the mandrel 302 may be integral with and/or coupled to the casing 30, such that the outer surface 316 (e.g., outer annular surface) of the mandrel 302 forms the outer diameter 31 of the casing 30 (e.g., at least along the length 314 of the mandrel 302). That is, the mandrel 302 may encapsulate a portion of the casing 30 and may be configured to increase a diameter of a portion of the casing 30, such that the recess 308 may be formed along the casing 30. In certain embodiments, the mandrel 302 may correspond to the sections 32 of the casing 30 illustrated in FIG. 1. For example, in certain embodiments, the mandrel 302 may be configured to align with the sealing layers 20 of the geological formation 12, such that the monitoring line 42 is integrated with the casing 30 for a distance that extends along the sealing layers 20. In certain embodiments, the length 316 of the mandrel 302 (e.g., from the first end 304 to the second end 306) may be selected and/or designed based on a dimension 315(e.g., length, depth) of the sealing layers 20. That is, the length 314 of the mandrel 302 may be substantially similar to the depth 315 of the sealing layers 20.

By employing the monitoring system 300 (e.g., mandrel 302 having the recess 308, the centralizers 324, and the binding agent 310), the monitoring lines 42 may be secured within the mandrel 302 (e.g., within a central portion of the recess 308), thereby eliminating potential gaps (e.g., gaps 64) between the monitoring lines 42 and the outer diameter of the casing 30. Further, by securing the monitoring lines 42 within the casing 30 (e.g., within the mandrel 302), additional space within the annular space 15 (e.g., between the outer surface 316 of the mandrel 302 and the open hole 13) may be provided that enables cement to readily flow therethrough to seal the annular space 15. The outer surface 316 of the mandrel 302 along with the outer surface 354 of the binding agent 310 may also provide a continuous (e.g., uniform, smooth, flush) surface (e.g., surface 357), thereby improving the quality of a cement bond formed between the cement and the surface 357 of the mandrel 302. Further still, with the monitoring lines 42 secured within the mandrel 302, the monitoring lines 42 may be limited from moving about the annular space 15, such that an amount of interference with the flow of cement through the annular space 15 is reduced or limited, as discussed above. That is, by integrating the monitoring lines 42 within the mandrel 302, the monitoring lines 42 may extend through the annular space 15 in a predictable manner, thereby reducing bends and/or turns in the monitoring lines 42 that may otherwise affect or hinder cementing operations.

It should be appreciated that while FIG. 10 illustrates and/or describes the mandrel 302 as extending for a length 314 that is substantially similar to a dimension of the sealing layer(s) 20, in other embodiments, the mandrel 302 may extend for any length of the casing 30. For example, the mandrel 302 may extend along an entire length of the casing 30 and/or along a length of the casing that aligns with multiple different layers (e.g., injection layer 18, additional layers 22). That is, in certain embodiments, the mandrel 302 (e.g., sections 32) may extend beyond the sealing layers 20, such that the recess 308 defined by the mandrel 302 and the binding agent 310 also extends beyond the sealing layers 20. Further, while a single recess 308 is illustrated in FIGS. 10-13, it should be appreciated that the mandrel 302 may include any number (e.g., two, three, four, or more) of recesses 308 to receive any number (e.g., one, two, three, four, or more) of monitoring lines 42. That is, the mandrel 302 may include a single recess 308 configured to receive any number (e.g., one, two, three, or more) of monitoring lines 42, or the mandrel 302 may include any number of recesses 308, where each recess 308 is capable of receiving any number (e.g., one, two, three, or more) of monitoring lines 42.

As noted above, in certain embodiments, each of the monitoring systems 100, 200, 300 discussed above (e.g., each of the mandrels 102, 202, 302 discussed above) may extend along and/or correspond to a section 32 of the casing 30 that aligns with the sealing layers 20 of the geological formation. In such embodiments, one or more portions of the monitoring line(s) 42 may not be contained and/or integrated with the casing 30. For example, in certain embodiments, portions of the monitoring line(s) 42 that align with the injection layer 18 and/or the additional layers 22 may not be integrated with the casing 30. In such embodiments, one or more of the monitoring systems 100, 200, 300 discussed herein may employ one or more spacers or clamps to ensure proper cementing conditions at least along the portions of the casing 30 in which the monitoring line(s) 42 are not integrated with the casing 30 (e.g., along the portions of the casing 30 that align with the injection layer 18 and/or additional layers 22).

For example, FIG. 14 is a schematic view of an embodiment of a clamp 400 configured to engage (e.g., grasp, retain, clamp) the monitoring line(s) 42. The clamp 400 may include a retaining portion 402 and an extension portion 404. The retaining portion 402 may be configured to retain and/or hold the monitoring line 42, and the extension portion 404 may couple the retaining portion 402 to the casing 30. For example, the extension portion 404 may be coupled to the casing 30 and may extend from the casing 30 (e.g., extend radially away from the casing 30 relative to a central axis of the casing 30) into the annular space 15 for a distance 405. In certain embodiments, the extension portion 404 may be configured to offset (e.g., space) the monitoring line 42 from the outer diameter 31 of the casing 30 by the distance 305. For example, a gap 408 may be provided between the monitoring line 42 and the outer diameter 31 of the casing 30 via the clamp 400, where a dimension (e.g., size, width, radial dimension) of the gap 408 is substantially similar to the distance 405. Notably, the extensions 404 may be configured such that a size of the gap 408 is greater than a size of the gaps 64 discussed above, thereby enabling cement to flow readily through the gap 408. That is, the distance 405 of the extension 404 may be selected such that the gap 408 is large enough to enable cement to flow readily through the gap 408 (e.g., through the gap at a desired mass flow rate). For example, in certain embodiments, the extension 404 may be designed, selected, and/or configured such that the gap 408 has a threshold radial dimension of at least one and a quarter centimeters (e.g., 0.5 inches), thereby enabling cement to readily flow therethrough.

Additionally, in certain embodiments, the retaining portion 402 of the clamp 400 may be configured to maintain tension within the monitoring line(s) 42, such that the monitoring line(s) 42 extend along the injection well 14 in a predictable manner. For example, the clamp 400 may be configured to reduce slack in the monitoring line(s) 42, such that bends and/or turns within the monitoring line(s) are reduced. A reduction in bends and turns in the monitoring lines 42 improves cementing operations, as discussed above.

It should be appreciated that while the discussion above focuses on carbon dioxide storage operations, the monitoring systems discussed herein may be employed with any type of well. For example, the monitoring systems discussed herein may be employed with other types of injection wells configured to inject fluids into a geological formation for storage. Additionally, or alternatively, the monitoring systems discussed herein may be employed with geothermal wells, hydrocarbon wells, and/or other types of producing wells, whereby the monitoring systems monitor the geological formations through which the wells extend.

Technical effects of the present disclosure include systems and methods that improve cementing conditions along an injection well and/or increase an integrity of a storage site. For example, the monitoring systems discussed herein may employ one or more mandrels extending along a length of a casing, whereby the one or more mandrels define one or more cavities, recesses, and/or passages configured to receive a monitoring line of the monitoring system. In certain embodiments, the monitoring systems may also employ a binding agent configured to be positioned within the cavities or recesses defined by the mandrel to retain the monitoring line within the mandrel. The mandrel and/or the binding agent may secure the monitoring line within the casing, such that the monitoring line extends through an annular space between the casing and an open hole in a predictable manner. Further, the mandrel and/or binding agent may provide a smooth, continuous, or uniform surface that facilitates a secure bond between cement directed through the annular space and the casing (e.g., the mandrel). That is, by integrating and/or incorporating the monitoring lines within the casing (e.g., within the mandrel), and by providing a smooth or continuous surface along an outer diameter of the casing, cementing operations along the annular space may be improved. In turn, an integrity of the storage site may be increased.

The subject matter described in detail above may be defined by one or more clauses, as set forth below.

A monitoring system for monitoring a geological formation includes a casing of an injection well, wherein the casing is configured to extend through an open hole of the geological formation to define an annular space between the casing and the open hole, and wherein the casing includes a mandrel circumscribing at least a portion of the casing, and one or more monitoring lines configured to monitor the geological formation for the presence of carbon dioxide, wherein the one or more monitoring lines are at least partially integrated with the mandrel.

The monitoring system of the preceding clause, wherein the geological formation includes a storage layer configured to receive the carbon dioxide from the injection well, and at least one sealing layer configured to limit migration of the carbon dioxide out of the storage layer into the at least one sealing layer.

The monitoring system of any preceding clause, wherein the mandrel is configured to align with the at least one sealing layer such that a length of the mandrel is equal to or greater than a dimension of the at least one sealing layer.

The monitoring system of any preceding clause, wherein the mandrel defines a passage configured to receive the one or more monitoring lines.

The monitoring system of any preceding clause, including a fitting positioned at an end of the mandrel, wherein the fitting is configured to centralize the monitoring line within the passage and seal the passage.

The monitoring system of any preceding clause, including a binding agent, wherein the mandrel defines a recess configured to receive the binding agent, and the recess and the binding agent are configured to collectively retain the monitoring line within the mandrel

The monitoring system of any preceding clause, wherein the binding agent includes an insert.

The monitoring system of any preceding clause, wherein the insert and the recess of the mandrel collectively define a cavity configured to receive the one or more monitoring lines, and wherein an outer surface of the insert and an outer surface of the mandrel are flush with one another in an assembled configuration of the monitoring system.

The monitoring system of any preceding clause, wherein the binding agent comprises an injectable binding agent configured to cure after a threshold amount of time.

The monitoring system of any preceding clause, including one or more centralizers configured to centralize the one or more monitoring lines within the recess, wherein each of the one or more centralizers includes a port, and the one or more monitoring lines extend through the port in an assembled configuration of the monitoring system.

The monitoring system of any preceding clause, wherein the one or more centralizers are configured to space the one or more monitoring lines a distance from a surface of the recess to form a gap between the one or more monitoring lines and the surface of the recess, wherein the injectable binding agent is configured to flow through the gap.

A monitoring system for a well includes one or more monitoring lines configured to monitor the well, a mandrel configured to circumscribe a casing of the well, wherein the mandrel defines a recess, and an insert configured to cooperate with the recess of the mandrel to retain the one or more monitoring lines within the mandrel.

The monitoring system of the preceding clause, wherein the recess includes a first surface configured to engage with a first surface of the insert, a second surface configured to engage with a second surface of the insert, a third surface configured to engage with a third surface of the insert, and a fourth surface configured to engage with a fourth surface of the insert.

The monitoring system of any preceding clause, wherein the recess includes a fifth surface extending between the third surface and the fourth surface of the recess, wherein the fifth surface is configured to align with a fifth surface of the insert to collectively define a cavity configured to receive the one or more monitoring lines.

The monitoring system of any preceding clause, wherein the mandrel includes an outer surface, the insert includes a sixth surface, and wherein the outer surface of the mandrel and the sixth surface of the insert are aligned within one another to form a flush surface along an outer diameter of the mandrel in an assembled configuration of the monitoring system.

The monitoring system of any preceding clause, including one or more clamps positioned above the mandrel, wherein the one or more clamps are configured to space the one or more monitoring lines from an outer diameter of the casing and maintain tension within the one or more monitoring lines.

A monitoring system for a well includes one or more monitoring lines configured to monitor the well, a mandrel configured to circumscribe a casing of the well, wherein the mandrel defines a recess, one or more centralizers positioned within the recess and configured to centralize the one or more monitoring lines within the recess, and an injectable binding agent configured to be injected into the recess to retain the one or more monitoring lines within the recess of the mandrel.

The monitoring system of the preceding clause, wherein the monitoring system includes a casing strap having a body defining one or more ports distributed along a length of the casing strap, wherein the one or more ports are configured to align with the recess of the mandrel during assembly of the monitoring system, and wherein the casing strap is configured to fluidly couple to a binding agent source to inject the injectable binding agent into the recess via the one or more ports.

The monitoring system of any preceding clause, wherein each of the one or more centralizers includes a body portion, a port extending through the body portion, wherein the one or more monitoring lines extend through the port in an assembled configuration of the monitoring system, one or more extensions extending from the centralizer and configured to engage with one or more surfaces of the recess.

The monitoring system of any preceding clause, wherein the centralizer is configured to offset the one or more monitoring lines from the one or more surfaces of the recess.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. Moreover, the order in which the elements of the methods described herein are illustrated and described may be re-arranged, and/or two or more elements may occur simultaneously. The embodiments were chosen and described in order to best explain the principals of the disclosure and its practical applications, to thereby enable others skilled in the art to best utilize the disclosure and various embodiments with various modifications as are suited to the particular use contemplated.

Finally, the techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

1. A monitoring system for monitoring a geological formation, the monitoring system comprising:
a casing of an injection well, wherein the casing is configured to extend through an open hole of the geological formation to define an annular space between the casing and the open hole, and wherein the casing comprises a mandrel circumscribing at least a portion of the casing; and
one or more monitoring lines configured to monitor the geological formation for the presence of carbon dioxide, wherein the one or more monitoring lines are at least partially integrated with the mandrel.

2. The monitoring system of claim 1, wherein the geological formation comprises a storage layer configured to receive the carbon dioxide from the injection well, and at least one sealing layer configured to limit migration of the carbon dioxide out of the storage layer into the at least one sealing layer.

3. The monitoring system of claim 2, wherein the mandrel is configured to align with the at least one sealing layer such that a length of the mandrel is equal to or greater than a dimension of the at least one sealing layer.

4. The monitoring system of claim 1, wherein the mandrel defines a passage configured to receive the one or more monitoring lines.

5. The monitoring system of claim 4, comprising a fitting positioned at an end of the mandrel, wherein the fitting is configured to centralize the monitoring line within the passage and seal the passage.

6. The monitoring system of claim 1, comprising a binding agent, wherein the mandrel defines a recess configured to receive the binding agent, and the recess and the binding agent are configured to collectively retain the monitoring line within the mandrel.

7. The monitoring system of claim 6, wherein the binding agent comprises an insert.

8. The monitoring system of claim 7, wherein the insert and the recess of the mandrel collectively define a cavity configured to receive the one or more monitoring lines, and wherein an outer surface of the insert and an outer surface of the mandrel are flush with one another in an assembled configuration of the monitoring system.

9. The monitoring system of claim 6, wherein the binding agent comprises an injectable binding agent configured to cure after a threshold amount of time.

10. The monitoring system of claim 9, comprising one or more centralizers configured to centralize the one or more monitoring lines within the recess, wherein each of the one or more centralizers comprises a port, and the one or more monitoring lines extend through the port in an assembled configuration of the monitoring system.

11. The monitoring system of claim 10, wherein the one or more centralizers are configured to space the one or more monitoring lines a distance from a surface of the recess to form a gap between the one or more monitoring lines and the surface of the recess, wherein the injectable binding agent is configured to flow through the gap.

12. A monitoring system for a well, comprising:
one or more monitoring lines configured to monitor the well;
a mandrel configured to circumscribe a casing of the well, wherein the mandrel defines a recess; and
an insert configured to cooperate with the recess of the mandrel to retain the one or more monitoring lines within the mandrel.

13. The monitoring system of claim 12, wherein the recess comprises:
a first surface configured to engage with a first surface of the insert;
a second surface configured to engage with a second surface of the insert;
a third surface configured to engage with a third surface of the insert; and
a fourth surface configured to engage with a fourth surface of the insert.

14. The monitoring system of claim 13, wherein the recess comprises a fifth surface extending between the third surface and the fourth surface of the recess, wherein the fifth surface is configured to align with a fifth surface of the insert to collectively define a cavity configured to receive the one or more monitoring lines.

15. The monitoring system of claim 14, wherein the mandrel comprises an outer surface, the insert comprises a sixth surface, and wherein the outer surface of the mandrel and the sixth surface of the insert are aligned within one another to form a flush surface along an outer diameter of the mandrel in an assembled configuration of the monitoring system.

16. The monitoring system of claim 12, comprising one or more clamps positioned above the mandrel, wherein the one or more clamps are configured to:
space the one or more monitoring lines from an outer diameter of the casing; and
maintain tension within the one or more monitoring lines.

17. A monitoring system for a well, comprising:
one or more monitoring lines configured to monitor the well;
a mandrel configured to circumscribe a casing of the well, wherein the mandrel defines a recess;
one or more centralizers positioned within the recess and configured to centralize the one or more monitoring lines within the recess; and
an injectable binding agent configured to be injected into the recess to retain the one or more monitoring lines within the recess of the mandrel.

18. The monitoring system of claim 17, wherein the monitoring system comprises a casing strap having a body defining one or more ports distributed along a length of the casing strap, wherein the one or more ports are configured to align with the recess of the mandrel during assembly of the monitoring system, and wherein the casing strap is configured to fluidly couple to a binding agent source to inject the injectable binding agent into the recess via the one or more ports.

19. The monitoring system of claim 17, wherein each of the one or more centralizers comprises:
a body portion;
a port extending through the body portion, wherein the one or more monitoring lines extend through the port in an assembled configuration of the monitoring system; and
one or more extensions extending from the centralizer and configured to engage with one or more surfaces of the recess.

20. The monitoring system of claim 19, wherein the centralizer is configured to offset the one or more monitoring lines from the one or more surfaces of the recess.
